(19) United States
(12) Patent Application Publication
(10) Pub. No.: US 2010/0305101 A1
(43) Pub. Date: Dec. 2, 2010

(54) Leptin genotype and ß-adrenergic agonists

(76) Inventor: Foley Leigh Shaw Marquess, Saskatoon (CA)

Correspondence Address: RICHES, MCKENZIE & HERBERT, LLP, SUITE 1800, 2 BLOOR STREET EAST, TORONTO, ON, M4W 3J5, CA

(21) Appl. No.: 12/662,668

(22) Filed: Apr. 28, 2010

Related U.S. Application Data
(60) Provisional application No. 61/213,132, filed on May 8, 2009.

(51) Int. Cl. A61K 31/55; A61K 31/137; A61P 3/00; A61P 21/06; C12Q 1/68

(52) U.S. Cl. 514/214.02; 514/653; 435/6

(57) ABSTRACT

A method of identifying livestock animal subgroups of the same species, from a group of livestock animals of the same species wherein the subgroup has similar genetic predispositions for response to Zilpaterol Hydrochloride (ZH) treatment with respect to marbling, HCW gain, REA size gain, DDMI, % EBF, and YG's. The genetic potential of each animal to respond to ZH treatment is established by determining the LeptinArg25Cys genotype and segregating individual animals into subgroups based upon the LeptinArg25Cys genotype.

### SUMMARY OF THE INVENTION

The present invention relates to genotyping animals for the leptin Arg25Cys functional mutation and also application of a class of compounds known as β-adrenergic agonists (β-AA), specifically Zilpaterol Hydrochloride (ZH), and Ractopamine Hydrochloride (RH), in order to take advantage of newly observed interactions between the leptin genotype and β-AA's on phenotypes; namely hot carcass weight (HCW) gain, body fat gain, rate of fat gain, marbling score, quality and yield grade, size of eye area (REA), percent empty body fat (% EBF), and daily dry matter intake (DDMI). Through knowledge of leptin genotype we can more precisely apply β-AA's yielding optimized HCW response of specific genotypes, reduced or no reduction in marbling score or carcass quality grades, reductions in REA, improvements in DDMI consistency, and improvements in % EBF consistency in comparison to mass application of β-AA's. The present invention allows for precise and specific administration of specific β-AA's to leptin genotype subgroups of animals.

### BACKGROUND OF THE INVENTION

### General β-Adrenergic Agonists (β-AA) Summary

A class of compounds known as β-adrenergic agonists (β-AA) has been used in the livestock industry as repartitioning agents. These phenethanolamine compounds promote the deposition of lean muscle tissue at the expense of adipose tissue by shifting nutrient use toward carcass lean tissue deposition and away from adipose tissue (3). β-agonists are used to produce maximum lean tissue growth, improved efficiency of gain and maximum feed efficiency in livestock (6). Two β-AA's that are commercially available to beef producers are Zilpaterol Hydrochloride (ZH) and Ractopamine Hydrochloride (RH).

β-adrenergic agonists are a class of organic molecules, which bind to β-adrenergic receptors (β-AR). β-AR's are present on the surface of most mammalian cells. There are three subtypes of β-AR's: β-AR₁, β-AR₂ and β-AR₃. The physiological response of a cell to a β-AA is specific to the combination of the three subtypes present on that cell (2). The distribution of receptor subtypes and the proportion of each subtype vary between tissue types in a species as well as between species. β-AR's are stimulated by the neurotransmitter norepinephrine and the medullary hormone epinephrine. β-AA's are administered orally to cattle, pigs, poultry and sheep in order to increase muscle accretion and decrease adipose tissue accretion (2).

β-AA's bind to the G-coupled protein β-adrenergic receptors, which releases the Gₛ protein into the cell (2). The Gₛ protein's alpha subunit activates the enzyme adenylylcyclase, which converts ATP to cyclic-adenosine monophosphate (cAMP), a major intracellular signaling molecule. Increased intracellular concentration of the secondary messenger cAMP causes the activation of Protein Kinase A (PKA). PKA goes on to phosphorylate many intracellular proteins. Two of the targets of phosphorylation by PKA are hormone sensitive lipase which is responsible for the rate limiting step in adipocytetriacylglycerol degradation and acetyl-CoAcarboxylase which is the rate limiting enzyme in long chain fatty acid synthesis (2). Hormone sensitive lipase is activated by phosphorylation by PKA to stimulate breakdown of triacylglycerols in adipose tissue. Acetyl-CoAcarboxylase is inactivated by phosphorylation by PKA which inhibits fatty acid synthesis. As a result of treatment with β-AA we expect the inhibition of lipogenesis and the stimulation of lipolysis in adipose tissue. PKA also phosphorylates and activates the cAMP response element binding protein (CREB), a transcription factor that is both a positive and a negative regulator of gene transcription. CREB is located in the nucleus, bound to the cAMP response element which is located in the regulatory element of various genes. CREB's phosphorylation by PKA can either up or down regulate expression of various genes (2).

The first β-AA used in the cattle industry was Clenbuterol (7). Clenbuterol was shown to decrease fat mass and increase weight gain and gain-to-feed ratio in livestock. Much remains unknown about the exact mechanism of action of β-AA's. The mechanism is complicated as a result of both cellular and systemic effects. Furthermore, many effects from β-AA's seen in vivo are not always replicated in vitro revealing that the effects at the cellular level may be linked to other effects in the animal. Treatment of animals with β-AA's lead to increased muscle mass, which may be due to either an increase in muscle protein synthesis, a decrease in muscle protein degradation or a combination of the two. The increase in lean tissue is due to muscle hypertrophy as there is no increase in the amount of DNA present in the skeletal muscle tissue (3). β-AA treatment results in increased mRNA transcripts for several muscle proteins such as the myosin light chain and alpha-actin and also increased mRNA transcripts of the calpain protease inhibitor calpastatin (3). Both β-AA's RH and ZH are applied in the last 20 to 42 days on feed in cattle (6). Over a course of 3 to 5 weeks of treatment with β-AA skeletal muscle is unable sustain this increased level of fiber hypertrophy without additional DNA and responsiveness to the β-adrenergic agonists is dampened and the response decreases with time.

β-AA's also act on β-adrenergic receptors (β-AR's) located in adipose tissue. Through phosphorylation, which activates hormone sensitive lipase, β-AA's stimulate adipocytetriacylglycerol degradation. Through the phosphorylation, which inactivates acetyl-CoAcarboxylase, β-AA's inhibit fatty acid and triacylglycerol synthesis. The β-AA's RH and ZH stimulate the lipolytic system and increase the plasma concentration of nonesterified fatty acids in animals undergoing treatment with β-AA's (2). β-AA's have also been shown to reduce expression of lipogenic genes (4). The net result of treatment with phenethanolamine repartitioning agents such as RH and ZH are increased protein accretion and decreased rate of fat deposition.

β-AA also exerts effects outside of the direct binding to the β-AR on skeletal muscle and adipose tissue. Other mechanisms which, may contribute to increased skeletal muscle accretion include the binding of β-AA's to β-AR's on the smooth muscle surrounding arteries and blood vessels. Treatment with β-AA's causes vasodilation which increase circulation to skeletal muscle and adipose tissue. Increased blood flow to skeletal muscle may also enhance muscle hypertrophy by delivering increased amounts of substrates and energy sources for amino acid uptake and protein synthesis (2). Increased blood flow to adipose tissue due to vasodilation may carry away nonesterified fatty acids and increase lipolysis. Mechanisms of action of β-AA may also include involvement of secondary hormones whose release is controlled by β-AR present on skeletal muscle and adipose tissue. It has also been suggested that β-AA can cross the blood-brain barrier and act directly on the central nervous system to control feed intake (2).

The effects of β-agonist vary between species. This may be because some animals have less potential for increased growth because they are closer to the biological maximum growth rate (example: broiler chickens) (2).

### Summary of Zilpaterol Hydrochloride (ZH)

Zilpaterol Hydrochloride is a β-agonist, which was shown to bind to both the β-AR₁ and β-AR₂ receptors (5). ZH is marketed in North America under the Zilmax® trade name and is manufactured by Intervet Schering-Plough Animal Health (Millsboro, Del.). ZH produces similar results in livestock as its sister phenethanolamine compound RH. Much is still unknown regarding the mechanism by which ZH improves lean tissue deposition and increases feed efficiency in cattle. Treatment with ZH decreases the cellular levels of tumor necrosis factor alpha (TNF-α) and increases the cellular levels of cAMP mainly through the β-AR₂ receptors (5). Similar to RH, treatment of ZH results in increased levels of cAMP and inhibition of proteolysis in muscle tissue, stimulation of lipolysis and reduction of lipogenesis.

Studies comparing RH and ZH have been conducted (6). Although the patents state that ZH binds to β2 adrenergic receptors it also binds to β1. In addition, ZH has anti-inflammatory properties. In one study RH was feed at a 300 mg/steer/day and ZH was fed at 6 mg/steer/day (6). Animals fed ZH had larger LM (Longissimus Muscle) area, but RH had no effect. Comparisons to non-treated steers showed a HCW increase of 14 kgs (30.8 lbs) and 22 kgs (48.5 lbs) for RH and ZH, respectfully (6). Both RH and ZH increased sheer force and increased toughness of the muscle tissue (6).

ZH, or Zilmax®, administered to cattle has been shown to increase HCW, final body weight, dressing percentage; and reduce subcutaneous fat (12^{th} rib fat), marbling score and USDA quality grade (17,18, & 20). It is generally understood that administration of ZH does not reduce daily dry matter intake (DDMI) of feed (20).

### Summary of Ractopamine Hydrochloride (RH)

Ractopamine Hydrochloride (RH) is one β-AA, which is shown to increase protein accretion and increase growth of livestock (6). RH is marketed in North America under the Optaflexx® trade mark and is manufactured by Elanco Animal Health (Greenfield, Ind.). RH binds with a higher affinity to β-AR₂. RH exists as four stereoisomers. The ranked order of affinities for the β-AR agonists are RR>RS>SR>SS. RH acts as a β-AR agonist in adipose tissue which results in an increase in plasma concentration of free fatty acids through the previously mentioned mechanisms. Swine fed RH have a reduced percentage of carcass fat but the rate of fat accretion is not consistently reduced. This effect is short live in adipose tissue because B-AR's are down-regulated by nearly 50% within the first 7 days of treatment with RH (8). Receptor down regulation may be responsible for the limited effectiveness of RH on adipose tissue. RH binds to the β-AR and increases the levels of intracellular cAMP. There is a direct link between cAMP and the transcriptional regulation for myosin heavy chain and bovine calpastatin (4). β-AA's have also been shown to activate other signaling pathways (such as the MAP kinase pathway) in common with insulin. Insulin promotes protein synthesis and inhibits protein degradation.

The β-adrenoceptor coupled adenylatecyclase system response has shown two different ways of desensitization to β-agonists. The first response is called heterologous desensitization in which there is a decrease in the cellular response to the original agonist. The second pattern is homologous desensitization, which is considered to be a refractory phase to the original β-agonist or similar compounds.

There are many possible explanations to homologous desensitization showing a decrease in β-adrenergic receptors in the presence of β-agonists over long periods. β-adrenergic receptors down regulation is accomplished through sequestering, internalization or removal of receptors from the cell surface to be degraded. They suggest intermediate feeding of RH. Although in swine a decrease of β-adrenergic receptors in adipose tissue has been observed there is no decrease of β-adrenergic receptors in skeletal muscle with prolonged RH treatment. There is a decreased response of muscle accretion in muscle after 4 weeks but not receptor response. The increase of muscle hypertrophy is due to in muscle α-actin synthesis and decreased activity of calpastatin (19).

RH, or Optaflexx®, like ZH has been shown to improve ADG (average daily gain), G:F (gain to feed, feed conversion), and hot carcass weight (HCW) gain (6 & 21).

### Leptin Hormone and Genotyping Summary

Leptin is a 16 kDa protein transcribed from the obese gene in mammals. Leptin is mainly produced by and secreted from white adipose tissue. Leptin acts on central as well as peripheral tissues to regulate feed intake, energy expenditure and whole body energy balance (9). Leptin is involved in a feedback regulatory loop. Leptin acts as a sensor, monitoring the level of energy stores which are indicated by the size of the adipose tissue mass. Circulating leptin communicates this information to the appetite center at the hypothalamus. Once leptin is released by the adipose tissues it circulates in the bloodstream to the brain where it binds to the hypothalamic center which receives and processes the intensity of the leptin signal through leptin receptors. The binding of leptin to its receptors in the hypothalamus effects numerous systems including the sympathetic nervous system to control the two main determinants of energy balance: feed intake and energy expenditure. When functioning under ideal conditions, this feedback regulatory loop serves to maintain a constant body weight. Leptin production is increased following weight gain in order to decrease feed intake and increase metabolism, whereas leptin plasma levels decrease following weight loss in order to increase appetite and decrease metabolism. Chronic administration of leptin to ob/ob mice, which lack leptin due to a mutation in the obese gene, causes the animals to lose weight and to maintain their weight loss. Levels of leptin are increased in ruminants with increased body fat and/or energy balance (14).

There is a single nucleotide polymorphism (SNP) in the bovine leptin sequence which has a phenotypic affect on the animal. A cytosine to thyamine substitution in exon 2 of the bovine leptin gene encodes an amino acid transition of Arginine to Cysteine (Arg25Cys) (12). In the mature leptin protein, this amino acid change is located at the fourth amino acid position from the N-terminus of the molecule. A signal peptide on the immature protein (1st to 21st amino acids) is cleaved off before leptin is excreted from adipose tissue (12). Additionally the T allele in the obese gene, which causes the Arg to Cys transition, causes a structural change due to an alteration in disulfide bonding which in turn affects carcass level of fatness, yield grade, and quality grade (11). There is a disulphide bond between cysteine 96 and cysteine 146 which appears to be important for structure folding and receptor binding because a mutation of either of the cysteines renders the protein biologically inactive (15). It has been hypothesized that in TT animals, the Arg25Cys SNP disrupts the disulfide bond between Cys 96 and Cys 146 disrupting leptin's secondary and tertiary structure and altering the ability of leptin to bind to its receptor.

The Arg to Cys transition in animals homozygous for the T allele (TT) are believed to possess impaired leptin function, binding and recognition of leptin by the leptin receptors at the hypothalmus. In turn these animals are thought to show increased fat deposition and have higher levels of leptin mRNA (10). It is thought because leptin is not recognized at the receptor level, the signal to decrease appetite and increase metabolism is not delivered. The Arg25Cys transition has been associated with higher levels of fat deposition in beef cattle (10). There is a positive correlation between serum leptin concentration with insulin, live and carcass weight, days on feed as well as a negative correlation with lean meat yield (10). A positive correlation exists between serum leptin levels and quality grade. TT animals have higher levels of circulating leptin and have increased fat deposition compared to CC animals (22).

Circulating leptin binds on two families of hypothalamic neurons to the leptin receptor. The result of leptin binding to the first population of hypothalamic neurons is reduced expression of neuropeptide Y (NPY) and agouti-related peptide (AGRP). NPY and AGRP are both orexigenic (feed inducing) molecules therefore their down-regulation reduces appetite. When Leptin binds to the second population of receptors it induces the expression of two anorexigenic (feed inhibiting) neuropeptides: α-melanocyte-stimulating hormone (α-MSH) which is derived from pro-opiomelanocortin (POMC) and cocaine and amphetamine-related transcript (CART). α-MSH is an agonist of the melanocortin-4 receptor (MC4R), which reduces food intake when activated. Leptin also stimulates the release of corticotropin releasing hormone (CRH) which also upregulates POMC. The expression of leptin therefore induces a reduction in food intake through the suppression of orexigenic neuropeptides and the induction of anorexigenic neuropeptides (13).

The LeptinArg25Cys SNP has been demonstrated to impact carcass backfat level, live animal backfat level, marbling score, Canada and USDA Yield Grade, Canada and USDA Quality Grade, and rate of backfat accretion over time (11; Cactus Trial—08-01). Specifically, TT animals (animals homozygous for the T allele) have more carcass backfat, live animal backfat, and marbling than CT or CC animals respectively. As well, TT animals have been shown to have an increased probability of being scored yield grade (YG) 3 (Canada) and 4 (USDA) as compared to CT and CC animals, respectively; and TT animals have been shown to have a decreased amount of YG1 (Canada and USDA) as compared to CT and CC animals, respectively when slaughtered on the same day and all environmental factors being the same. Since the base associations and effects of the genotypes have been established, producers can now manipulate the days on feed (DOF) of feedlot animals in order to optimize these characteristics, i.e. shorten DOF of TT's in order to increase the percent of YG 1's and maintain marbling level; and/or lengthen the DOF of CT and CC animals in order to increase the level of marbling in the animals without negatively impacting the YG.

Current application of β-AA's

Currently β-AA's (either ZH or RH) are mass applied to pens of animals contained in one feedlot. Optaflexx® (RH) is registered in Canada and the USA for feeding during the 28-42 days prior to slaughter, with no withdrawal time required; and Zilmax® (ZH) is registered for feeding during the 20-40 days prior to slaughter with a three day withdrawal. No categorical identification of animals has been identified or used in differential application of ZH or RH. Although conceivable, targeted β-AA administration in the same feedlot, in practice would be unusual. Further, currently it is not conceivable of any method of administering a β-AA other than mass application of one β-AA in one feedlot to a pen. Currently there is no evidence of an effective system of categorizing animals such that multiple or selective β-AA application is advantageous. Currently there is no effective system, which identifies an animals' genetic propensity to respond to β-AA application. Currently there is no effective system, which sorts pens of animals based on a categorical identifying system, such as leptin genotype, and selectively apply the β-AA's to each subgroup. Therefore, multiple β-AA feeding in the same feedyard based on leptin genotype would be a novel change to the current β-AA application strategy.

### SUMMARY OF THE INVENTION

### Objectives

An objective of this invention is to select which cattle receive a β-AA, or if a β-AA should be administered, based on leptin genotype and which β-AA, if any, is then administered.

An objective of this invention is to select which β-AA is to be administered (i.e. ZH or RH) to animals based upon leptin genotype.

An objective is to administer two or more β-AA's in the same feedlot based on leptin genotype.

An objective of this invention is to select which leptin genotype does not receive a β-AA.

An objective of this invention is to manage marbling and quality grade by differential β-AA application to specific leptin genotypes.

An objective of this invention is to manage HCW gain response by differential β-AA application to specific leptin genotypes.

An objective of this invention is to manage REA size gain response by differential β-AA application to specific leptin genotypes.

An objective of this invention is to manage DDMI by differential β-AA application to specific leptin genotypes.

An objective of this invention is to manage yield grade and backfat by differential β-AA application to specific leptin genotypes.

An objective of this invention is to manage % EBF response by differential β-AA application to specific leptin genotypes.

Yet a further object of the invention is to determine what the leptin genotype is in order to determine the genetic propensity for daily dry matter intake, and rates of back fat accretion.

### ADVANTAGES

The process of genotyping for the LeptinArg25CysSNP allows feedlot operators to identify animals by their genotype and their individual genetic potential for optimal responses to specific β-AA administration. This will give producers more knowledge that will allow them to be more informed about the decision process related to β-AA administration. The nature of the genetic propensity of each of the different genotypes will help feedlots more accurately characterize the projected animal response, based at least in part by genotype. This allows producers or owners of animals to make more informed decisions and take appropriate actions with respect to each of the genotype groups, i.e. control and manage the application of each β-AA differentially to different genotype groups, or have no β-AA administered. These actions will yield more predictable outcomes for the producers, and will include outcomes such as improving the consistency and response of DDMI, HCW gain, REA size, marbling and quality grades, and backfat and yield grades. It will also spare financial resources that would be expended upon animals whose response to β-AA results in very little economic value.

### Impacts of β-Adrenergic Agonist and Leptin Genotyping

Producers will benefit from the integration of leptin genotyping and administration of β-adrenergic agonists by the identification and application of the interaction knowledge. Knowledge of leptin genotype will allow sub-grouping of animals for specific application of specific β-adrenergic agonists to specific genotype sub groups, or no application of β-AA's to certain genotypes. These interaction benefits include:
1. When β-AA's are selectively administered specific leptin genotype sub groups (CC's and CT's) having increased hot carcass weight gain as compared to other specific genotype sub groups (TT's).
2. When β-AA's are selectively administered to specific leptin genotype subgroups (CC's) having no reduction in quality grade or marbling score as compared to other specific genotype subgroups (CT's and TT's).
3. When β-AA's are selectively administered to specific leptin genotype subgroups (TT's) having reduced size of rib eye area gain and smaller overall piece size as compared to other specific genotype subgroups (CT's and TT's).
4. When β-AA's are selectively administered to specific leptin genotype subgroups (CT's and TT's) having a reduction in DDMI during β-adrenergic agonist administration as compared to other genotype subgroups (CC's).
5. When β-AA's are selectively administered to specific leptin genotype subgroups (CT's and TT's) having specific β-adrenergic agonist applied in order to avoid a reduction in daily dry matter intake during β-adrenergic agonist administration period as compared to other specific genotype subgroups (CC's).
6. When β-AA's are selectively administered to specific leptin genotype subgroups having differential β-adrenergic agonists administered in order to optimize rate of back fat accretion and days on feed to optimal slaughter date as compared to other specific genotype subgroups.
7. When β-AA's are selectively administered to specific leptin genotype subgroups will not receive β-adrenergic agonist administration while other specific genotype subgroups will receive β-adrenergic agonist administration.
8. When β-AA's are selectively administered to specific leptin genotype subgroups (TT's) have a larger reduction in % EBF as compared to other specific genotype subgroups (CC's and CT's).

Further examples of systems which take advantage of the present invention are as follows:

A system comprising ZH administration to only CC animals in order to avoid the adverse effect of reduced marbling in the CT and TT animals, optimize hot carcass weight gain (the largest in CC animals), optimize rib eye area gain (the smallest in CC animals), and not suffer the adverse effects of reduced dry matter intake during ZH administration in the CT and TT animals. Another system comprises of CC animals receiving ZH administration along with a subgroup of the CT animals which would optimize hot carcass weight gain response along with marbling response in those animals which are most probable candidates for ZH treatment so as to avoid excessive HCW gain which would result in final HCW which is above 453.7 kg (1000 lbs), or any weight which results in a discount from slaughter houses for excessive weight. Another system comprises TT animals not receiving any ZH treatment and receiving either no β-adrenergic agonist treatment or alternatively RH treatment in order to optimize the marbling response of animals and avoid any adverse consequences of ZH treatment, and potentially receive the weight gain benefits from RH treatment. Another system comprises feeding all animals ZH except black hided TT's and/or black hided CT's in order to allow animals to express their maximum genetic potential for marbling, which will increase the probability of reaching the Certified Angus Beef Quality standards. This same system would have the black hided TT and/or CT animals receive either RH treatment or no β-adrenergic agonists treatment.

A system comprising β-adrenergic agonist administration to only CC animals in order to avoid the adverse effect of reduced marbling in the CT and TT animals. Another system comprises of CC animals receiving RH administration along with a subgroup of the CT animals which would optimize hot carcass weight gain response along with marbling response in those animals which are most probable candidates for RH treatment so as to avoid excessive HCW gain which would result in final HCW which is above 453.7 kg (1000 lbs), or any weight which results in a discount from slaughter houses for excessive weight. Another system comprises TT animals not receiving any RH treatment and receiving no β-adrenergic agonist treatment in order to optimize the marbling response of animals and avoid any adverse consequences of RH treatment. Another system comprises feeding all animals RH except black hided TT's and/or black hided CT's in order to allow animals to express their maximum genetic potential for marbling, which will increase the probability of reaching the Certified Angus Beef Quality standards. This same system would have the black hided TT and/or CT animals receive no β-adrenergic agonists treatment.

These benefits will optimize the financial outcomes of feeding cattle for slaughter. By applying either different β-adrenergic agonists to different genotype subgroups or no β-adrenergic agonists to specific genotypes, potential adverse effects of β-adrenergic agonist administration to whole populations will be avoided and positive effects will be accentuated by the more precise application of β-adrenergic agonists.

To this end, in one of its aspects, the invention provides a method for identifying livestock animal subgroups of the same species, from a group of livestock animals of the same species wherein the subgroup has similar genetic predispositions for response to Zilpaterol Hydrochloride (ZH) treatment with respect to marbling, HCW gain, REA size gain, DDMI, % EBF, and YG's comprising: (a) determining genetic potential of each animal to respond to ZH treatment by determining the LeptinArg25Cys genotype; and (b) segregating individual animals into subgroups based upon the LeptinArg25Cys genotype.

In yet another of its aspects, the invention provides a method of producing subgroups of animals based on their Leptin R25C genotype in order to optimize ZH treatment, whereby genotype subgroups either receive ZH treatment—or either no ZH treatment or RH treatment; to capitalize on the known LeptinArg25Cys genotype interactions with ZH treatment for the phenotypes of marbling score, stamped Quality Grades, REA size gain, HCW gain, DDMI (daily dry matter intake), and % EBF.

A further aspect of the invention includes a method of producing subgroups of animals based on their Leptin R25C genotype in order to optimize ZH treatment, whereby genotype subgroups either receive ZH treatment—or either no ZH treatment or RH treatment; to capitalize on the known LeptinArg25Cys genotype interactions with ZH treatment for the phenotypes of marbling score, stamped Quality Grades, REA size gain, HCW gain, DDMI (daily dry matter intake), and % EBF.

### BRIEF DESCRIPTION OF THE DRAWINGS

FIG. 1 is a schematic depiction of the trial design.

FIG. 2 illustrates the main effect of leptin genotype on carcass backfat level.

FIG. 3 illustrates backfat deposition rates by genotype (no Zilpaterol).

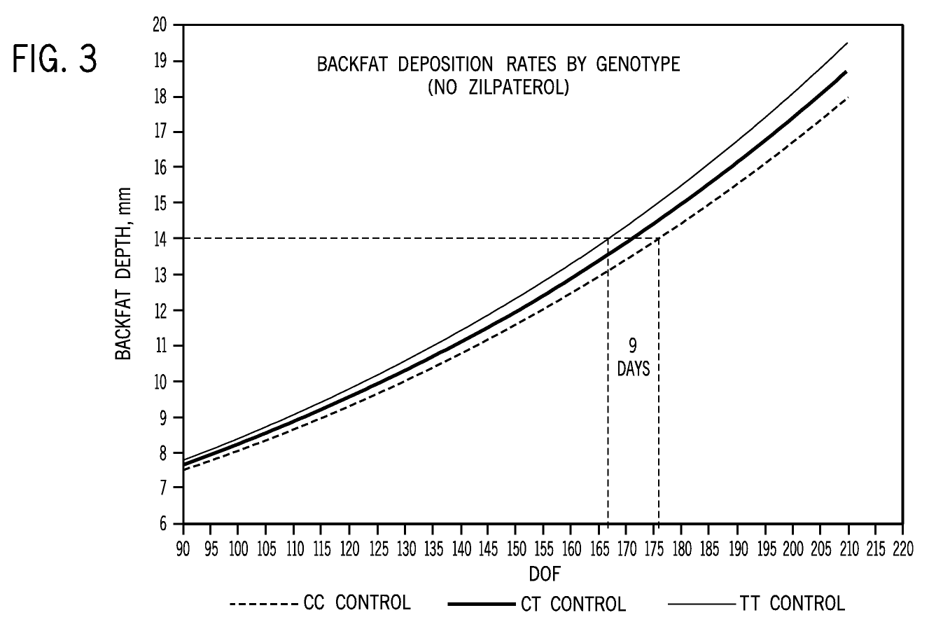

FIG. 4 visually describes the interaction between leptin genotype and ZH administration on USDA stamped quality grades Choice & Prime (Choice+Prime % by Zilpaterol and Leptin Genotype).

FIG. 5 visually describes the interaction between leptin genotype and ZH administration on marbling score (Marbling Score by Zilpaterol and Leptin Genotype (Small 0=400)).

FIG. 6 visually describes the interaction between leptin genotype and ZH administration on HCW gain (HCW Gain (lb): Genotype×Zilpaterol Tendency).

FIG. 7 visually describes the interaction between leptin genotype and ZH administration on daily dry matter intake during the 24 days preceding slaughter (21 days ZH administration+three days withdrawal)(Zilmax Feeding Period DMI, lb (last 24 days on feed): Genotype×Zilpaterol).

FIG. 8 illustrates the effect leptin genotype has on daily dry matter intake (DDMI) in the absence of any β-AA's.

FIG. 9 visually describes the interaction between leptin genotype and ZH administration on REA size gain (Rib Eye Area (in²): Genotype×Zilpaterol Tendency).

FIG. 10 visually describes the interaction between leptin genotype and ZH administration on % empty body fat (% EBF).

FIG. 11 illustrates leptin genotype and % EBF on and off ZH.

### DETAILED DESCRIPTION

In a trial completed at a private research facility in Texas, USA (Cactus Research, Amarillo Tex.) leptin genotype was assessed for potential interaction with Zilpaterol Hydrochloride (ZH). The trial consisted of 4,279 animals and occurred from summer of 2008 and carried through to spring of 2009. The trial was conducted as a randomized complete block design with approximately 90 animals being placed into pens based on leptin genotype and randomly assigned to drug treatment with pen being the experimental unit. Treatment structure was a 3×2 factorial including three letting genotypes (CC, CT and TT) and two drug treatments (zero control and drug treatment). Pens were blocked by time, specifically arrival date of the animal to the feedlot. Each block was slaughtered on the same day, and the complete process replicated eight times resulting in eight blocks. Below is a schematic summary of the trial design.

Upon arrival into the feedlot animals were individually weighed and back fat was measured using ultrasound. These measured were also taken 65, days on feed, and then one week prior to ZH treatment and 2-3 days prior to slaughter. All cattle were slaughtered and USDA carcass data were measured. Growth data were fitted to a non-linear growth model.

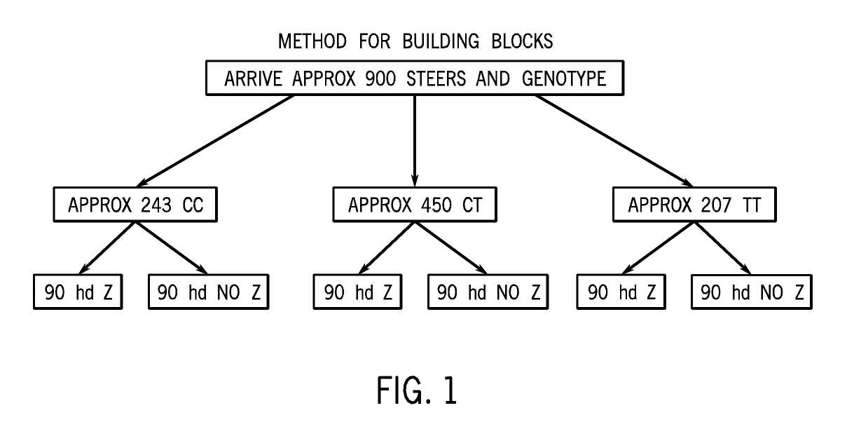

FIG. 1 illustrates a schematic summary of the trial design.

Backfat was measure by ultrasound at:
1) Arrival
2) 65 days on feed
3) 1 week prior to zilpaterol initiation
4) 2-3 days prior to slaughter
   8 total blocks, 6 treatment pens per block, and 4,179 head total (avg initial wt=875 lbs) Within blocks, all treatments were killed on the same day (avg days on feed=129

Results:

Leptin genotype did affect some response variables independent of ZH administration. For some response variables though, response to ZH administration was dependent upon leptin genotype and interactions were observed.

### Main Effect of Leptin Genotype on Carcass Back Observed:

TABLE 1
Carcass Backfat Depth (Leptin Main Effect Overall P = 0.01)

CC = 11.9 mm (0.47″)
CT = 12.2 mm (0.48″)
TT = 12.7 mm (0.50″)

### Main Effect Final Ultrasound Backfat Observed:

TABLE 2
Ultrasound Backfat Depth two days prior to slaughter
(Leptin Main Effect Overall P < 0.01)

CC = 11.2 mm (0.44″)
CT = 11.4 mm (0.45″)
TT = 11.7 mm (0.46″)

### Main Effect of Leptin Genotype Backfat Gain Observed:

TABLE 3
Overall Ultrasound Backfat Gain
(Leptin Main Effect Overall P = 0.03)

CC = 7.63 mm (0.30″)
CT = 7.88 mm (0.31″)
TT = 8.11 mm (0.32″)

### Main Effect of Leptin Genotype—Rate of Fat Deposition Observed:

TABLE 4
Overall Rate of Ultrasound Backfat Deposition
(Leptin Main Effect Overall P = 0.11)

CC = 0.0073 mm/d (0.0002874″/d)
CT = 0.0075 mm/d (0.0002952″/d)
TT = 0.0077 mm/d (0.0003031″/d)

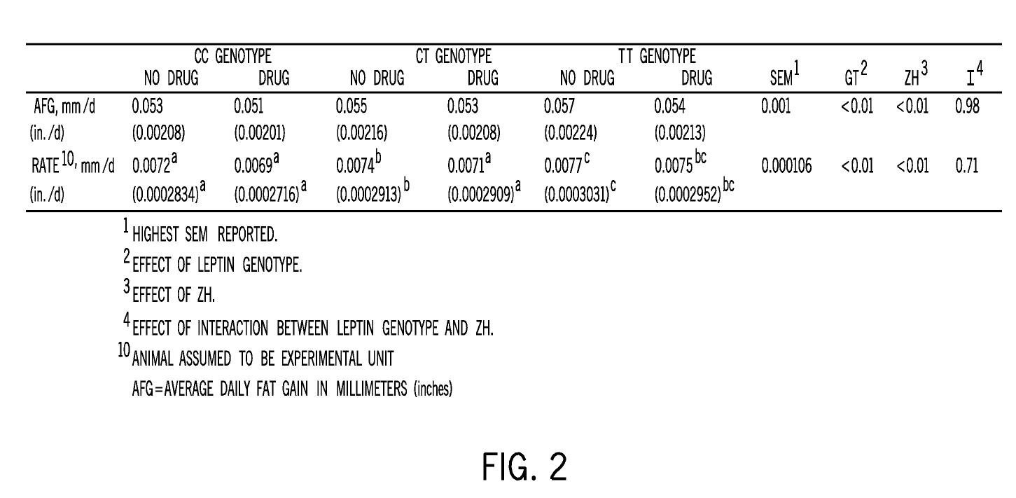

FIG. 2 illustrates the rates of backfat accretion by genotype (control only).

### Main Effect of Leptin Genotype on USDA YG 4

TABLE 5
YG 4 Frequency (Leptin Main Effect Overall P = 0.015)

CC = 2.7%
CT = 3.0%
TT = 5.3%

### Main Effect of Leptin Genotype on USDA YG 1

TABLE 6
YG 1 Frequency (Leptin Main Effect Overall P < 0.01)

CC = 26.4%
CT = 18.7%
TT = 17.7%

### Interactions

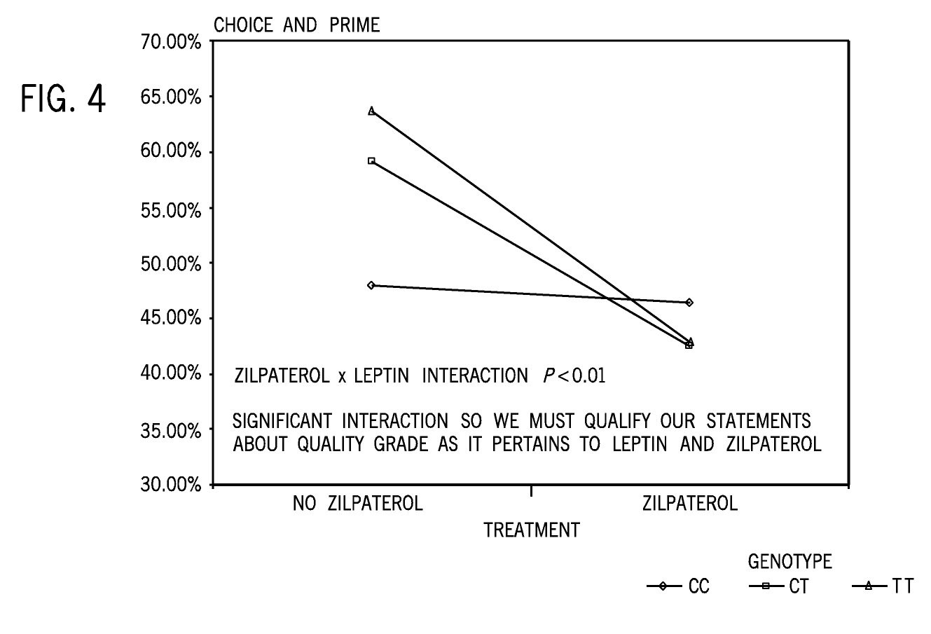

FIG. 4 illustrates the interaction between leptin genotype and ZH on USDA stamped quality grades Choice+Prime.

ZH administration significantly interacted with leptin genotype (P<0.01). As measured by USDA stamped quality grade categories, Choice+Prime, TT animals had the highest % Choice+Prime, CT animals were intermediate and CC animals had the lowest Choice+Prime without ZH administration. In animals administered ZH, CC were unaffected with respect to % Choice+Prime but TT and CT animals had significant reductions in % Choice+Prime. See FIG. 4.

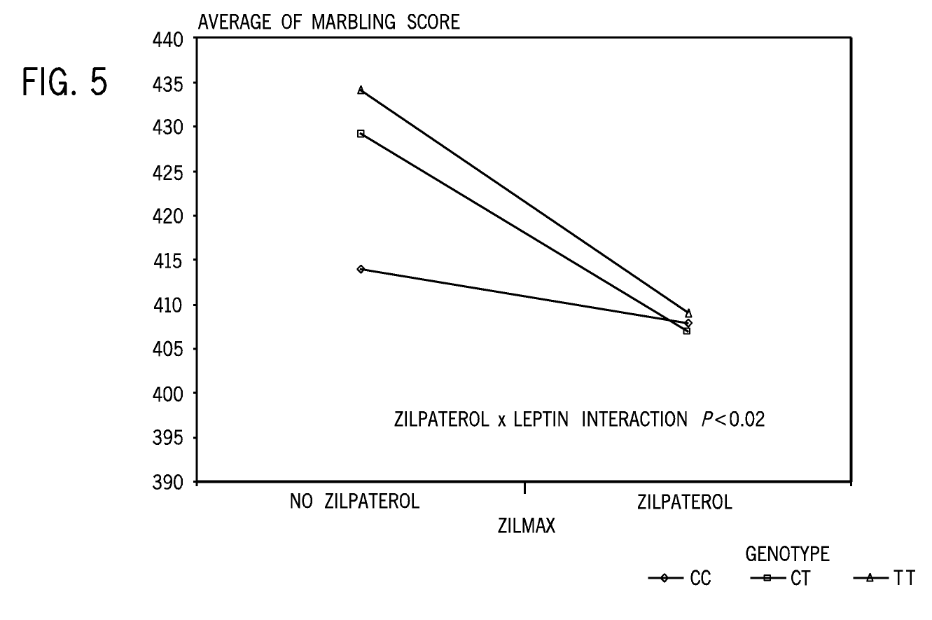

FIG. 5 illustrates the interaction between leptin genotype and ZH on marbling score.

ZH administration significantly interacted with leptin genotype (P<0.02) as measured by marbling score. CC's administered ZH had only a slight reduction in marbling score where as TT's and CT's administered ZH had a much greater reduction in marbling score. See FIG. 5.

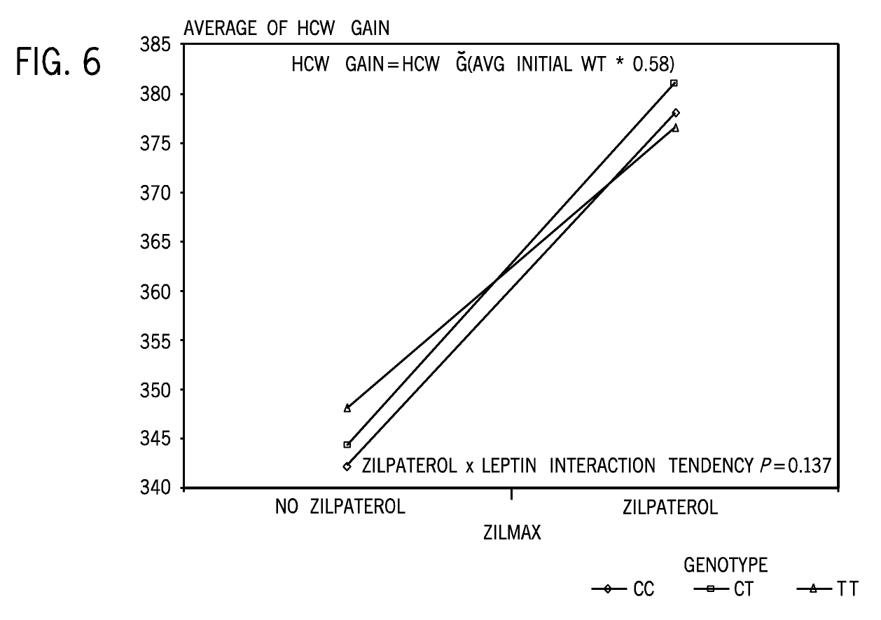

FIG. 6. Interaction between leptin genotype and ZH on HCW gain.

ZH administration has a statistical tendency to interact with leptin genotype (P=0.14) with respect to hot carcass weight gain. Response to ZH administration varied by genotype with the TT's having the lowest response to ZH administration. CC's and CT's have the largest response to ZH administration. There is a 3.4 kg (7.4 lb) HCW response difference between CC and TT animals (P<0.10). See FIG. 6.

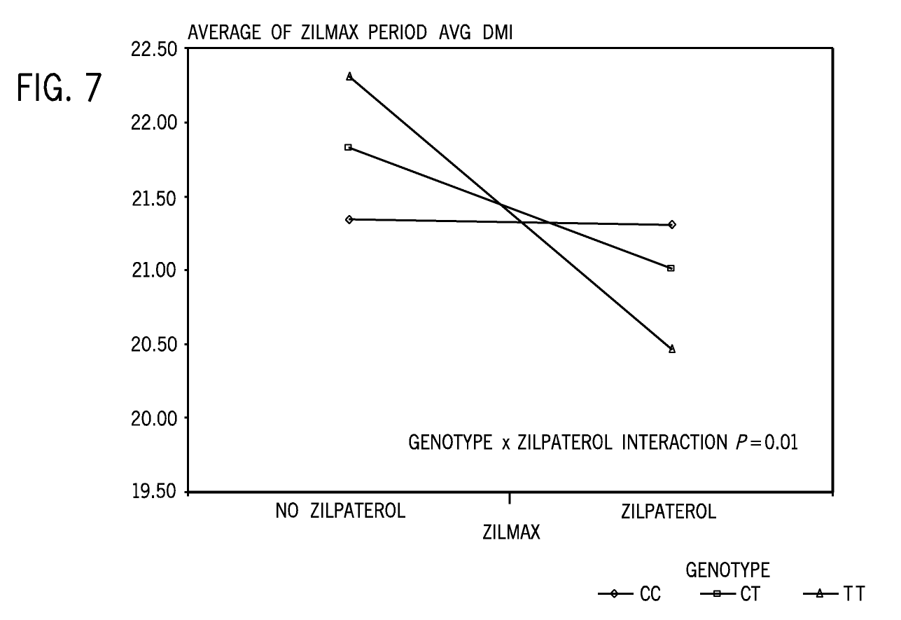

FIG. 7 illustrates the interaction between leptin genotype and ZH on daily dry matter intake (DDMI).

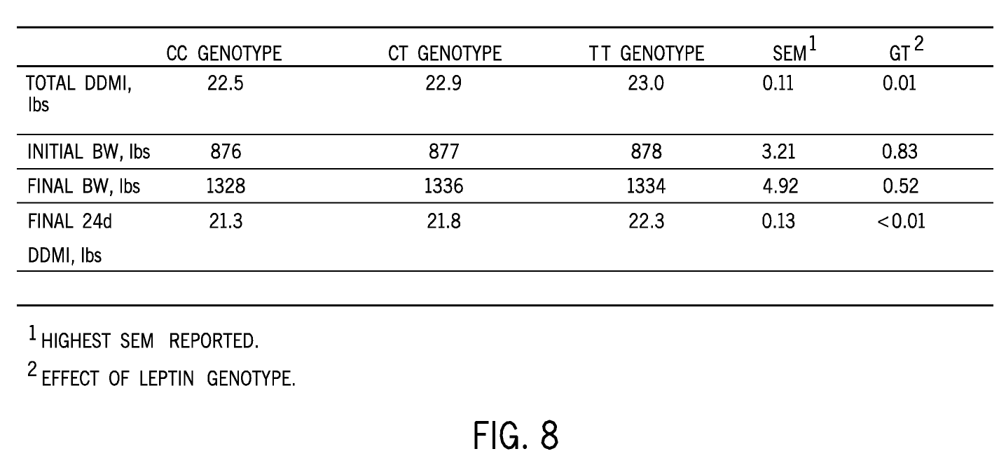

ZH administration has a statistically significant interaction with leptin genotype (P=0.01) with respect to daily dry matter intake (DDMI). In the absence of ZH administration, TT's had the highest DDMI, CC's had the lowest DDMI and the CT's consumed the intermediate amount (FIG. 8). When the TT's were administered ZH they had the lowest DDMI as compared to the CC's which had the highest DDMI. Again the CT's had an intermediate amount and had a DDMI lower than the CC animals. In summary, the CC animals had no change in DDMI during ZH treatment, but CT & TT animals had a significant reduction in DDMI during ZH treatment. See FIG. 7.

FIG. 8 illustrates the effect of leptin genotype on feed intake (DDMI).

Leptin genotype significantly impacted daily dry matter intake (FIG. 8). Specifically total DDMI was assessed for the complete feeding period, and the final 24 days of the feeding period. This assessment did not consider animals fed ZH or any interactions. Therefore, it only considered animals not fed β-AA's. TT animals have a significant increase in DDMI over the complete feeding period and the final 24 days on feed time period in comparison to CT and CC animals, respectively.

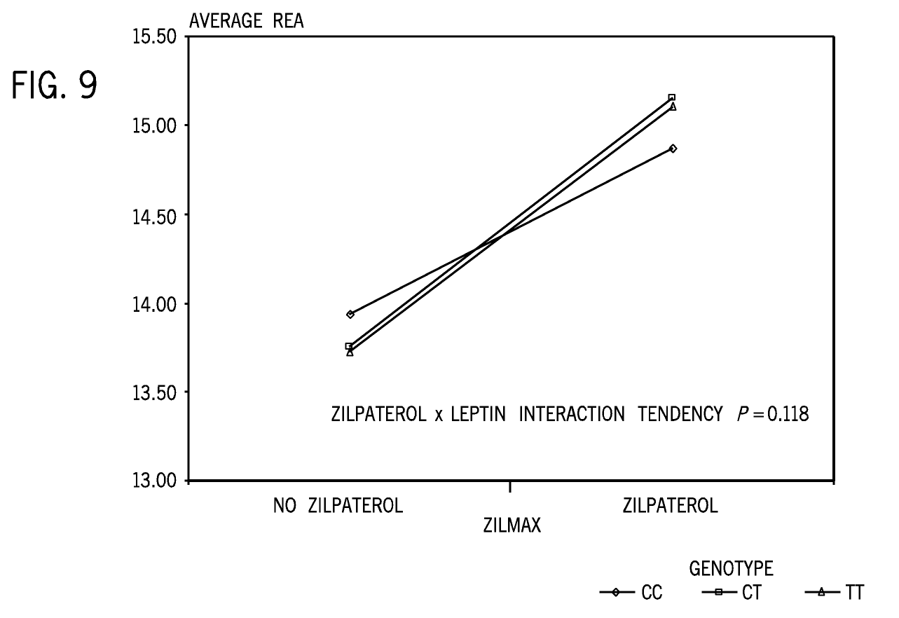

FIG. 9 illustrates the interaction between leptin genotype and ZH on size of rib eye area (REA) gain.

ZH administration has a statistical tendency to interact with leptin genotype (P=0.118) with respect to rib eye area (in²). Response to ZH administration varied by genotype with the CC's having the lowest response to ZH administration. TT's and CT's have the largest response to ZH administration. TT and CT animals had a gain of 1.4 square inches compared to CC's having a gain of 0.35 square cm's (0.9 square inches). See FIG. 9.

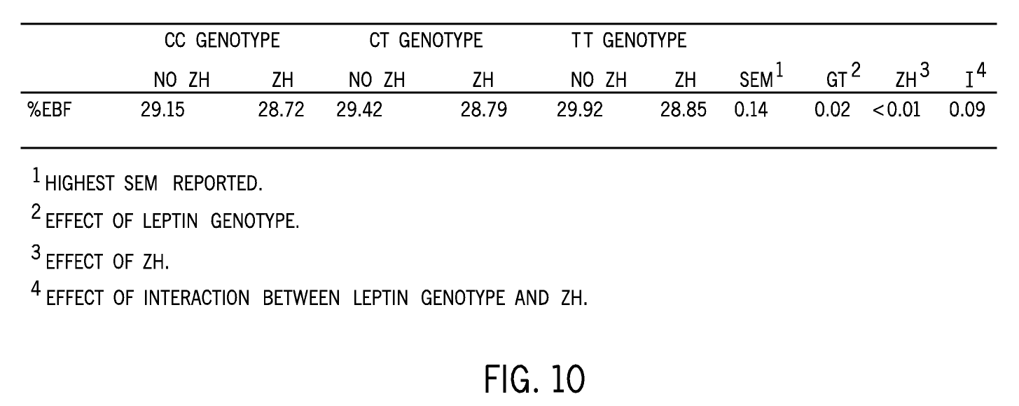

FIG. 10 illustrates the interaction between leptin genotype and ZH on % empty body fat (% EBF).

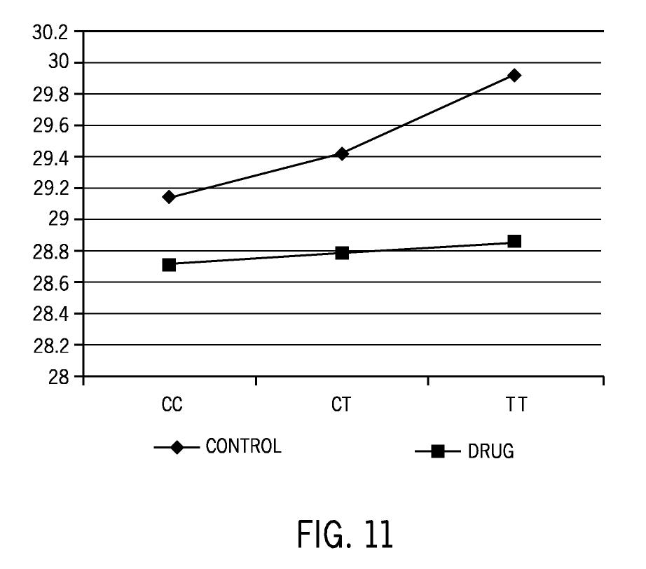

ZH administration tends to interact with leptin genotype (P=0.09) with respect to % EBF. Response to ZH administration varied by genotype with the CC's having the lowest response to ZH administration. TT's and CT's have the largest response to ZH administration. TT and CT animals respectively had the largest reductions in % EBF, with the TT animals having the largest reduction in % EBF. Since % EBF is a mathematical formula (23), which relies on the amount of marbling (quality grade), it is understood that the % EBF is in part a function of the marbling interaction between leptin genotype and ZH. As TT animals have the largest reduction in marbling when fed ZH it is no surprise that TT animals have the largest in % EBF when fed ZH. Concurrently, since CC animals experience no reduction in marbling when fed ZH it is very supportive that they too experience the smallest reduction in % EBF when fed ZH. See FIG. 11.

What was observed and discovered out of the trial work described herein is that mass application of Zilmax® and Optaflexx® is not necessary and does not yield optimal results. This is due the several interactions observed between leptin genotype and the β-AA's. These interactions teach that selective application of these growth promoting agents based on leptin genotype can yield results not obtained when the β-AA's are mass applied to pens of cattle.

Specifically, application of Zilmax® to CC genotype animals yields the most optimal results for this genotype. This is due to the larger than “label” or expected response in HCW, and small or no reduction in marbling and quality grade (USDA Choice or better). Marbling is an important attribute in carcass composition, and is commonly factored into how an animals' value is determined. Therefore, reaching a threshold amount of marbling is important to producers, and any factor that reduces the amount of marbling is a negative factor for producers; such as mass application of Zilmax®. In addition, REA size is optimal when it is kept to a size such that an acceptable portion size can be obtained, which in practice means that as the REA continues to get larger it is detrimental. Therefore, as Zilmax® is known to increase REA size, feeding Zilmax® to CC's can limit the downside in this area. Also, the detrimental effect of Zilmax® on % EBF is limited when fed to CC animals. And, importantly, no reduction in DDMI is observed when Zilmax® is fed to CC animals, which is contributing to the increased HCW observed in CC animals.

Conversely, when observing the TT animals fed Zilmax® it is clear that there are specific detrimental effects on important phenotypes. DDMI is reduced in TT animals fed Zilmax® in comparison to CT & CC animals, respectively, which is a contributor to the reduction in marbling and quality grades (USDA Choice or better). The reduction in DDMI in TT animals is detrimental to the economics of the animal as it increases its overall proportional maintenance cost. That is, as a proportion of the total energy available for gain, TT animals fed ZH have a smaller proportion out of their total energy intake per day than CC & CT animals. The reduction in marbling is also backed up by the fact that there is also the largest reduction in % EBF when TT animals are fed Zilmax® in comparison to CT & CC animals fed Zilmax®. Also, TT animals fed Zilmax® have the largest increase in REA size, which is detrimental to portion size acceptance. Also, importantly these same TT animals with reduced DDMI, marbling, quality grades, and increased REA size gain have a smaller than expected or label HCW gain when compared to CC & CT animals. This clearly has a negative impact on the value of feeding Zilmax® as producers are paid on the amount of HCW sold.

The process of genotyping each animal and determining their leptin genotype so that more homologous animals with respect to their leptin genotype can be grouped for selective β-AA feeding will yield improved biological results (DDMI, marbling, quality grades, HCW gain, and REA size gain), improved consumer friendly results, and improved economic results.

### REFERENCES

1. Marchant Forde, J. N., Lay J. R., D. C., Pajor, E. A., Richert, B. T., and A. P. Schinckel. The effects of ractopamine on behavior and physiology of finishing pigs. 2003. J. Anim. Sci. 81:416-422.
2. Mersmann, H. J. 1998. Overview of the effects of beta-adrenergic receptor agonists on animal growth including mechanisms of action. J. Anim. Sci. 76:160-172.
3. Johnson, B. J. and C. Y. Chung. 2007 The veterinary clinics of North America. Food animal Practice [0749-0720]. Alterations in the physiology of growth of cattle with growth enhancing components. 2007 vol:23 iss:2 pg:321-32, viii.
4. Mils, S. E. The biological basis for the Ractopamine Response, Journal of Animal Science. 2002.20:E28-E32.
5. Verhoeckx K C, Doornbos R P, van derGreef J, Witkamp R F, Rodenburg R J. 2005. Inhibitory effects of the beta-adrenergic receptor agonist zilpaterol on the LPS-induced production of TNF-alpha in vitro and in vivo. J Vet Pharm. Ther. 28(6):531-7.
6. Avendano-Reyes, L., Torres-Rodriguez, V., Meraz-Murillo, F. J., Perez-Linares, C., Figuearoa-Saavedra, F. and P. H. Robinson. 2006. Effects of two β-adrenergic agonists on finishing performance, carcass characteristics, and meat quality of feedlot steers. J. Anim. Sci. 84(12):3259-65.
7. Dalrymple, R. H., Baker, P. K., Gingher, P. E., Ingle, D. L., Pensack, J. M., and C. A. Ricks. 1984. A repartitioning agent to improve performance and carcass composition of broilers. J. Poult Sci. 63(12):2376-83.
8. Spurlock, M. E., Cusumano, J. C., Ji, S. Q., Anderson, D. B., Smith, C. K. 2nd, Hancock, D. L., and S. E. Mills. 1994. The effect of ractopamine on beta-adrenoceptor density and affinity in porcine adipose and skeletal muscle tissue. J. Anim. Sci. 72(1):75-80.
9. Houseknecht, K. L., Baile, C. A., Matteri, R. L., and M. E. Spurlock. 1998. The biology of leptin: a review. J. Anim. Sci. 76(5):1405-20.
10. Buchanan, F. C., Van Kessel, A. G., Boisclair, Y. R., Block, H. C. and J. J. McKinnon. The leptin ARg25Cys affects performance, carcass traits and serum leptin concentrations in beef. Can. J. of Anim. Sci. 87:153-156.
11. Kononoff, P. J., Deobald, H. M., Stewart, E. L., Laycock, A. D. and F. L. S. Marquess. 2005. The effect of a leptin single nucleotide polymorphism on quality grade, yield grade, and carcass weight of beef cattle. J. Anim. Sci. 83:927-932.
12. BUCHANAN, F. C., FITZSIMMONS, C. J., VAN KESSEL, A. G., THUE, T. D., WINKELMAN-SIM, D. C., and S. M. SCHMUTZ. 2002. Association of a missense mutation in the bovine leptin gene with carcassfat content and leptin mRNA levels. Genet. Sel. Evol. 34:105-116.
13. OUTER, E. 2002. Leptin Signaling, Adiposity, and Energy Balance. Annals of the New York Academy of Sciences. 967(1):379-388.
14. Ehrhardt, R. A., Slepetis, R. M., Siegal-Willott, J., Van Amburgh, M. E., Bell, A. W., and Y. R. Bosclair. 2000. Development of a specific radioimmunoassay to measure physiological changes of circulating leptin in cattle and sheep. J. Endocrinol. 166(3):519-28.
15. Zhang, F., Basinski, M. B., Beals, J. M., Briggs, S. L., Churgay, L. M., Clawson, D. K., DiMarchi, R. D., Furman, T. C., Hale, J. E., Hsiung, H. M., Schoner, B. E., Smith, D. P., Zhang, X. Y., Wery, J. P., and R. W. Schevitz. 1997. Crystal structure of the obese protein leptin-E100. Nature. May 8; 387(6629):206-9.
16. Kim, K. S., Larsen, N., Short, T., Plastow, G., and Rothschild, M. F. 2000. A missense variant of the porcine melanocortin-4 receptor (MC4R) gene is associated with fatness, growth, and feed intake traits. Mamm. Gen. 11:131-135.
17. Vasconcelos, J. T., Rathmann, R. J., Reuter, R. R., Leibovich, J., McMeniman, J. P., Hales, K. E., Covey, T. L., Miller, M. F., Nichols W. T., and M. L. Galyean. 2009. Effects of duration of zilpaterol hydrochloride feeding and days on the finishing diet on feedlot cattle performance and carcass traits. J Anim. Sci 86(8): 2005-2015.
18. Montgomery, J. L., Krehbiel, C. R., Cranston, J. J., Yates, D. A., Hutcheson, J. P., Nichols, W. T., Streeter, M. N., Bechtol, D. T., Johnson, E., TerHune, T., and T. H. Montgomery. 2009. Dietary zilpaterol hydrochloride. I. Feedlot performance and carcass traits of steers and heifers. J. Anim. Sci. 87:1374-1383.
19. Smith, S. B., D. K. Garcia, S. K. Davis, and D. B. Anderson. 1989. Elevation of a specific mRNA in longissimus muscle of steers fed ractopamine. J. Anim. Sci. 67:3495-3502.
20. Latest zilpaterol studies reviewed. 2010. K. S. Eng, J. Beckett, and J. Simpson. Feedstuffs. April 19.v82-No16.p 12.
21. Gruber, S. L., J. D. Tatum, T. E., Engle, M. A., Mitchell, S. B., Laudert, A. L., Schroeder, and W. J. Platter. 2007. Effects of ractopamine supplementation on growth performance and carcass characteristics of feedlot steers differing in biological type. J. Anim. Sci. 85:1809-1815.
22. Geary, T. W., McFadin, E. L., MacNeil, M. D., Grings, E. E., Short, R. E., Funston, R. N., and D. H. Keisler. 2003. Leptin as a predictor of carcass composition in beef cattle. J. Anim. Sci. 81:1-8.
23. Guiroy, P. J., Tedeschi, L. O., Fox, D. G., and J. P. Hutcheson. 2002. The effects of implant strategy on finished body weight of beef cattle. J. Anim. Sci. 80:1791-1800.

1. A method for identifying livestock animal subgroups of the same species, from a group of livestock animals of the same species comprising the subgroups, wherein the subgroup has similar genetic predispositions for response to Zilpaterol Hydrochloride treatment with respect to marbling, HCW gain, REA size gain, DDMI, % EBF, and YG's comprising: (a) determining genetic potential of each animal to respond to ZH treatment by determining the Leptin Arg25Cys genotype; and (b) segregating individual animals into subgroups based upon the Leptin Arg25Cys genotype.

2. The method of claim 1 further comprising collecting an assembly of animals of the subgroup and feeding such animals until the median body fat or median subcutaneous fat of individual animals of the subgroup is of the desired level; or similarly for subcutaneous fat level and/or a combination of such and body weight (live or carcass) is of a desired level.

3. A method of producing subgroups of animals based on their Leptin Arg25Cys genotype in order to optimize ZH treatment, whereby genotype subgroups either receive ZH treatment or either no ZH treatment or RH treatment; to capitalize on the known Leptin Arg25Cys genotype interactions with ZH treatment for the phenotypes of marbling score, stamped Quality Grades, REA size gain, HCW gain, DDMI (daily dry matter intake), and % EBF.

4. A method of selectively applying a combination of ZH, RH, and/or no β-AA based on an animals' leptin Arg25Cys genotype.

5. The method of claim 4 where the amount of marbling, as measured by quality grade or marbling score, is increased by administering leptin Arg25Cys TT animals RH; as compared to mass application of ZH to a pen of animals.

6. The method of claim 4 where the amount of marbling, as measured by quality grade or marbling score, is increased by administering leptin Arg25Cys TT animals no β-AA; as compared to mass application of ZH to a pen of animals.

7. The method of claim 4 where the amount of marbling, as measured by quality grade or marbling score, is increased by administering leptin Arg25Cys CT animals RH; as compared to mass application of ZH to a pen of animals.

8. The method of claim 4 where the amount of marbling, as measured by quality grade or marbling score, is increased by administering leptin Arg25Cys CT animals no β-AA; as compared to mass application of ZH to a pen of animals.

9. The method of claim 4 where the amount of marbling, as measured by quality grade or marbling score, is increased by administering only leptin Arg25Cys CC animals ZH; as compared to mass application of ZH to a pen of animals.

10. The method of claim 4 where the amount of HCW gain is increased by administering leptin Arg25Cys CC animals ZH; as compared to mass application of RH to a pen of animals.

11. The method of claim 4 where the amount of HCW gain is increased by administering leptin Arg25Cys CT animals ZH; as compared to mass application of RH to a pen of animals.

12. The method of claim 4 where the amount of HCW gain is increased by administering leptin Arg25Cys CC animals ZH; as compared to no β-AA being administered to a pen of animals.

13. The method of claim 4 where the amount of HCW gain is increased by administering leptin Arg25Cys CT animals ZH; as compared to no β-AA being administered to a pen of animals.

14. The method of claim 4 where the amount of REA size gain is decreased by administering leptin Arg25Cys TT animals RH; as compared to mass application of ZH to a pen of animals.

15. The method of claim 4 where the amount of REA size gain is decreased by administering leptin Arg25Cys TT animals no β-AA; as compared to mass application of ZH to a pen of animals.

16. The method of claim 4 where the amount of daily dry matter feed intake is increased by administering leptin Arg25Cys TT animals RH; as compared to mass application of ZH to a pen of animals.

17. The method of claim 4 where the amount of daily dry matter feed intake is increased by administering leptin Arg25Cys CT animals RH; as compared to mass application of ZH to a pen of animals.

18. The method of claim 4 where the amount of daily dry matter feed intake is increased by administering only leptin Arg25Cys CC animals ZH; as compared to mass application of ZH to a pen of animals.

19. The method of claim 4 where the amount of daily dry matter feed intake is increased by administering leptin Arg25Cys TT animals no β-AA; as compared to mass application of ZH to a pen of animals.

20. The method of claim 4 where the amount of daily dry matter feed intake is increased by administering leptin Arg25Cys CT animals no β-AA; as compared to mass application of ZH to a pen of animals.

21. The method of claim 4 where the amount of EBF is increased by administering leptin Arg25Cys TT animals RH; as compared to mass application of ZH to a pen of animals.

22. The method of claim 4 where the amount of EBF is increased by administering leptin Arg25Cys CT animals RH; as compared to mass application of ZH to a pen of animals.

23. The method of claim 4 where the amount of EBF is increased by administering leptin Arg25Cys TT animals no β-AA; as compared to mass application of ZH to a pen of animals.

24. The method of claim 4 where the amount of EBF is increased by administering leptin Arg25Cys CT animals no β-AA; as compared to mass application of ZH to a pen of animals.
